(19) United States
(12) Patent Application Publication
(10) Pub. No.: US 2025/0334657 A1
(43) Pub. Date: Oct. 30, 2025

(54) Lightweight Magnetic Resonance Imaging Systems With Improved Portability And Reduced Eddy Current Induction

(71) Applicant: Zepp Europe Holding B.V., Amsterdam- Duivendrecht (NL)

(72) Inventors: Kongqiao Wang, Hefei (CN); Haoyang Wang, Hefei (CN); Jinghua Wang, Hefei (CN); Wang Huang, Hefei (CN)

(21) Appl. No.: 18/651,272

(22) Filed: Apr. 30, 2024

(51) Int. Cl. G01R 33/383; A61B 5/055; G01R 33/385

(52) U.S. Cl. CPC G01R 33/383; A61B 5/055; G01R 33/385

(57) ABSTRACT

A magnetic resonance imaging (MRI) system that includes a support member and permanent magnets. The permanent magnets are positioned within the support member such that the permanent magnets are arranged along a single plane, which may be either linear or non-linear (e.g., curved), and vary in size (e.g., height and/or diameter).

### TECHNICAL FIELD

The present disclosure relates generally to magnetic resonance imaging (MRI) systems and, more specifically, to lightweight MRI systems that offer improved portability.

### BACKGROUND

MRI generates images that highlight differences between healthy and unhealthy tissue, which can be used to diagnose many diseases and abnormal body conditions (e.g., tumors, strokes, heart problems, spine diseases, etc.). MRI scans offer safer alternatives for medical imaging (e.g., in contrast to x-ray, computed tomography, and positron emission tomography) in that MRI does not subject patients and medical personnel to ionizing radiation exposure. During MRI scans, a powerful, constant magnetic field, rapidly changing local magnetic fields, radiofrequency (RF) energy, and dedicated equipment are used.

Every year, more than 35 million MRI scans are performed in the United States and more than 70 million MRI scans are performed worldwide. High-quality scans increase diagnostic sensitivity and accuracy and are generally characterized by a high signal-to-noise ratio, high contrast between normal and pathological tissues, low levels of artifacts, and appropriate spatial-temporal resolution.

In order to obtain a detectable magnetic resonance (MR) signal, the patient being examined is positioned in a homogeneous magnetic field so that the patient's nuclear spins generate net magnetization that is oriented along the magnetic field. The net magnetization is rotated away from the magnetic field using an RF excitation field with the same frequency as the Larmor frequency of the nucleus.

The angle of rotation is determined by the field strength of the RF excitation pulse and the duration thereof. At the end of the RF excitation pulse, the nuclei, in relaxing to their normal spin conditions, generate a decaying MR signal at the same radio frequency as the RF excitation. The MR signal is detected (collected) by a receiver coil and is then amplified and processed (e.g., via a computing device) to obtain an MR image. The acquired measurements, which may be collected in the spatial frequency domain, can be digitized and stored as complex numerical values in a k-space matrix, and the associated MR image can be reconstructed from the k-space data (e.g., via an inverse 2D or 3D fast Fourier transformation).

The current trend in clinical imaging has been to increase the field strength of MRI systems in order to improve image quality and efficiency (e.g., scan time, signal-to-noise ratio, temporal-spatial. resolution, contrast, etc.). Known MRI systems, however, are expensive to procure, operate, service, and maintain and typically include a ferromagnetic metallic frame (yoke), which significantly increases the weight of MRI systems, thus limiting availability and access to MRI scans.

As such, a need remains for lightweight, cost-effective MRI systems that increase portability and access to MRI scans, which is addressed by the present disclosure.

### SUMMARY

In one aspect of the present disclosure, an MRI system is disclosed that includes a support member and permanent magnets, which vary in size and are positioned within the support member such that the permanent magnets are arranged along a single plane.

In certain embodiments, the permanent magnets may be arranged such that the single plane is linear.

In certain embodiments, the permanent magnets may be arranged in concentric rings.

In certain embodiments, the permanent magnets may be arranged such that the single plane is a curved hyperplane.

In certain embodiments, the curved hyperplane may be hemispherical.

In certain embodiments, the curved hyperplane may be hemicylindrical.

In certain embodiments, the permanent magnets may increase in size with distance from the centerpoint of the support member.

In certain embodiments, the permanent magnets may increase in transverse cross-sectional dimension with distance from the centerpoint of the support member.

In certain embodiments, the permanent magnets may be generally uniform in height.

In certain embodiments, the permanent magnets may increase in height with distance from the centerpoint of the support member.

In another aspect of the present disclosure, an MRI system is disclosed that includes a support member, which includes a non-magnetic, low-conductivity material, and permanent magnets, which are positioned within the support member in a uniform, symmetrical distribution.

In certain embodiments, the permanent magnets may include generally annular transverse cross-sectional configurations.

In certain embodiments, the permanent magnets may vary in size.

In certain embodiments, the permanent magnets may vary in transverse cross-sectional dimension with distance from the centerpoint of the support member.

In certain embodiments, the permanent magnets vary in height with distance from the centerpoint of the support member.

In another aspect of the present disclosure, an MRI system is disclosed that includes: a first support member; first permanent magnets that vary in size and which are positioned within the first support member; a second support member that faces the first support member, wherein the first support member and the second support member each include a non-magnetic, low-conductivity material; and second permanent magnets that vary in size and are positioned within the second support member.

In certain embodiments, the first support member and the second support member may be generally identical in configuration.

In certain embodiments, the first permanent magnets and the second permanent magnets may be generally identical in configuration.

In certain embodiments, the first permanent magnets and the second permanent magnets may be non-identical in configuration.

In certain embodiments, the first permanent magnets may increase in transverse cross-sectional dimension with distance from the centerpoint of the first support member.

In certain embodiments, the second permanent magnets may increase in transverse cross-sectional dimension with distance from the centerpoint of the second support member.

In certain embodiments, the first permanent magnets may increase in height with distance from the centerpoint of the first support member.

In certain embodiments, the second permanent magnets may increase in height with distance from the centerpoint of the second support member.

In certain embodiments, the MRI system may further include a first gradient panel, which is secured to the first support member, and a second gradient panel, which is secured to the second support member.

In certain embodiments, the first gradient panel and the second gradient panel may each include at least one coil to support magnetic field generation.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a side, perspective view of an example MRI system in accordance with the principles of the present disclosure, which includes an (optional) frame and a plurality of discrete permanent magnets.

FIG. 2 is a partial, plan view of the frame.

FIG. 3 is a top, perspective view of the permanent magnets according to an alternate embodiment of the present disclosure in which the permanent magnets include generally annular (e.g., circular) transverse cross-sectional configurations.

FIG. 4 is a top, perspective view of the permanent magnets according to another embodiment of the present disclosure in which the permanent magnets vary in height.

FIG. 5 is a side, perspective view of the permanent magnets according to another embodiment of the present disclosure in which the permanent magnets are arranged in a generally spherical orientation.

FIG. 6 is a side, perspective view of the permanent magnets according to another embodiment of the present disclosure in which the permanent magnets are arranged in a generally cylindrical orientation.

FIG. 7 is a side, perspective view of another embodiment of the MRI system, which includes gradient panels.

FIG. 8 is a side, perspective view of the MRI system seen in FIG. 7 shown with a patient.

FIGS. 9 and 10 are side, perspective views of alternate embodiments of the MRI system seen in FIG. 7.

FIG. 11 is a flow diagram illustrating the use of an artificial intelligence (AI) network to optimize performance of the MRI system.

FIG. 12 is a flow diagram illustrating one example of a methodology in which the AI network seen in FIG. 11 is utilized to optimize performance of the presently disclosed MRI system.

### DETAILED DESCRIPTION

The present disclosure describes an MRI system that includes a frame and plurality of permanent magnets, which are configured as discrete, individual components of the MRI system. The frame includes (i.e., is formed from) a non-magnetic, low electrical conductivity material, which not only results in a lightweight, portable system, but inhibits (if not entirely prevents) eddy current induction. In certain embodiments it is envisioned that the non-magnetic, low electrical conductivity material(s) may also be non-metallic and/or have a high impedance.

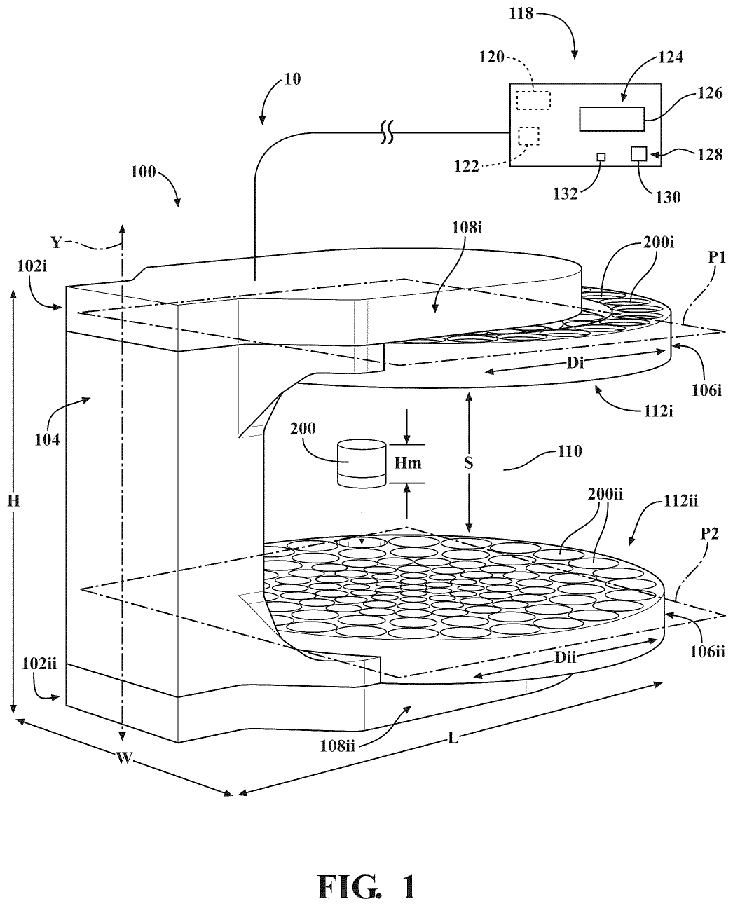

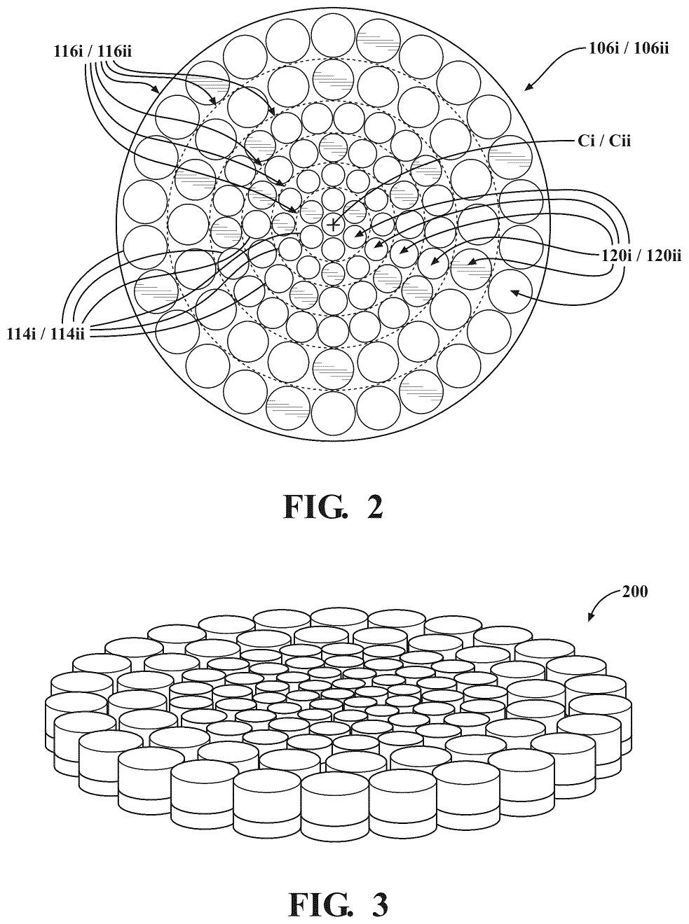

With reference to FIGS. 1 and 2, an example MRI system 10 is disclosed that includes a frame (yoke) 100 and a plurality of discrete permanent magnets (blocks) 200, which are configured as individual components of the MRI system 10. Although generally illustrated and described in the context of MRI herein below, it is envisioned that the principles of the present disclosure may find applicability to magnetic resonance spectroscopy (MRS) as well.

The frame 100 defines opposite ends 102i, 102ii and includes a generally C-shaped configuration. More specifically, in the illustrated embodiment, the frame 100 includes: an (optional) backspan 104, which defines (extends along) a longitudinal axis Y; (first, upper and second, lower) support members 106i, 106ii, which extend from the backspan 104 and are oriented in facing relation; and (first, upper and second, lower) support arms 108i, 108ii. In contrast to known MRI systems, which typically include a metallic frame that is formed from a ferromagnetic material (e.g., iron), the frame 100 includes (i.e., is formed from) one or more non-metallic, non-magnetic, low electrical conductivity, high-impedance materials (e.g., materials having a resistivity of at least 0.1 ohms/cm). More specifically, in the illustrated embodiment, the frame 100 includes (i.e., is formed from) one or more composite, high-impedance materials (e.g., carbon fiber). Embodiments of the MRI system 10 that incorporate one or more alternate materials into the frame 100 are also envisioned herein, however, and would not be beyond the scope of the present disclosure.

Embodiments of the MRI system 10 that are devoid of the backspan 104 are also envisioned, however, and would not be beyond the scope of the present disclosure. In such embodiments, it is envisioned that the support members 106i, 106ii may be supported in any manner suitable for the intended purpose of examining a patient in the manner described herein.

Although shown as including a pair of support members 106, embodiments including a single support member 106 are also envisioned herein (e.g., to facilitate the examination of a patient's prostate), as described in further detail below, and would not be beyond the scope of the present disclosure.

Forming the frame 100 (e.g., the support members 106) from the material(s) described herein imparts a variety of benefits to the MRI system 10 (i.e., vis-à-vis known MRI systems). For example, constructing the frame 100 in the manner described herein reduces the cost and the weight of the MRI system 10, which improves portability and facilitates robust usage thereof (e.g., whole-body scanning). For example, in the illustrated embodiment, the MRI system 10 includes a weight that lies substantially within the range of approximately 100 kg to approximately 500 kg (e.g., approximately 400 kg) and defines: a length L that lies substantially within the range of approximately 0.1 m to approximately 2 m (e.g., approximately 1 m); a width W that lies substantially within the range of approximately 0.1 m to approximately 1.5 m (e.g., approximately 0.75 m); and a height H that lies substantially within the range of approximately 0.2 m to approximately 2 m (e.g., approximately 1 m). Embodiments of the MRI system 10 in which one or more of the weight, the length L, the width W, and the height H may lie outside of the corresponding disclosed range are also envisioned herein (e.g., depending upon the particular intended use of the MRI system 10), however, and would not be beyond the scope of the present disclosure.

In contrast to known MRI systems, in which the metallic frame typically forms part of the magnetic circuit, the frame 100 inhibits (if not entirely prevents) eddy current induction, which not only improves the quality of the images that are generated by the MRI system 10, but reduces the load on the MRI system 10 and the complexity of the MRI system 10. For example, constructing the frame 100 from the material(s) described herein obviates the need for the eddy current countermeasures that are typically required in known MRI systems, which further reduces the cost, the weight, and the size of the MRI system 10.

The support members 106i, 106ii respectively receive (house) permanent magnets 200i, 200ii such that the permanent magnets 200i, 200ii are positioned within the support members 106i, 106ii. The permanent magnets 200i, 200ii are arranged along single planes P1, P2 (i.e., such that the plane P1 extends through each of the permanent magnets 200i and the plane P2 extends through each of the permanent magnets 200ii) in uniform, symmetrical distributions. More specifically, the permanent magnets 200i, 200i are spaced in a generally consistent and even manner from each other and are symmetrically distributed about (multiple) axes that extend in generally parallel relation to transverse cross-sectional dimensions (i.e., diameters) Di, Dii of the support members 106i, 106ii (and the planes P1, P2).

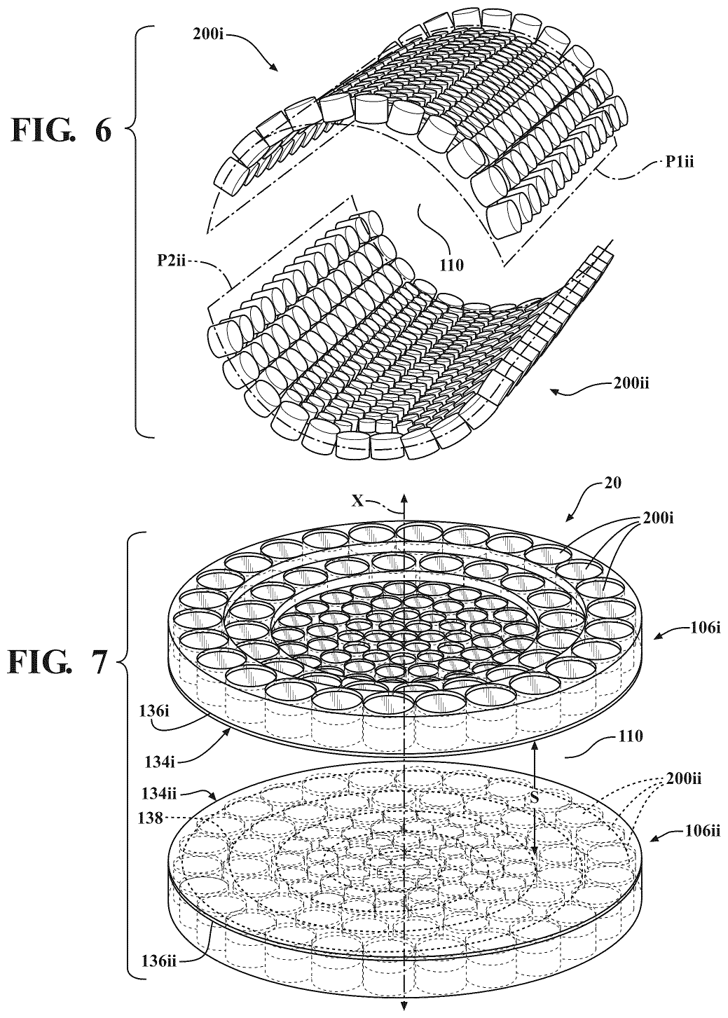

In the embodiment illustrated in FIG. 1, the support members 106i, 106ii are configured such that the planes P1, P2 are generally linear in configuration. Embodiments in which the planes P1, P2 may be non-linear (e.g., curved) are also envisioned herein (FIGS. 5, 6), as described in further detail below, and would not be beyond the scope of the present disclosure.

The support members 106i, 106ii are located at the ends 102i, 102ii of the frame 100, respectively, and are oriented in facing relation. More specifically, the support members 106i, 106ii are separated from each other along the longitudinal axis Y so as to define a diagnostic space 110 therebetween that is configured to receive a patient, which may be generally planar (FIG. 1), generally spherical (FIG. 5), generally cylindrical (FIG. 6), etc., depending upon the particular configurations of the support members 106i, 106ii. The support members 106i, 106ii define opposite magnetic poles 112i, 112ii, which, together with the permanent magnets 200, generate a magnetic field across the diagnostic space 110 and dictate the shape thereof.

In the illustrated embodiment, the MRI system 10 (e.g., the frame 100) is configured such that the support members 106i, 106ii are separated by a distance S (FIG. 1) that lies substantially within the range of approximately 200 mm to approximately 400 mm (e.g., 300 mm) in order to facilitate whole-body scanning. Embodiments in which the MRI system 10 may be configured such that the distance S lies outside of the disclosed range are also envisioned herein, however. For example, an embodiment in which the MRI system 10 may be configured such that the distance S is less than 200 mm (e.g., to facilitate more targeted scanning of a specific body part) would not be beyond the scope of the present disclosure.

In certain embodiments, it is envisioned that the MRI system 10 may include (or otherwise accommodate) a support surface (not shown) for the patient (e.g., such that the patient is supported along an axis that extends in generally orthogonal (perpendicular) relation to the direction of the magnetic field). In such embodiments, it is envisioned that the support surface may be either fixed or variable in configuration. For example, it is envisioned that the support surface may include an elevation adjustment mechanism to selectively adjust the height thereof (and the position of the patient) in relation to the frame 100.

The support members 106i, 106ii are generally identical in configuration and include generally annular (e.g., circular) transverse cross-sectional configurations. Embodiments in which the particular configurations of the support members 106i, 106ii may be varied are also envisioned herein, however, and would not be beyond the scope of the present disclosure.

The support member 106i extends from the backspan 104 in generally orthogonal (perpendicular) relation thereto and defines (first, upper) receptacles (chambers) 114i, which are configured to receive (first, upper) permanent magnets 200i (FIG. 1) such that the permanent magnets 200i are secured (embedded) within the support member 106i. The receptacles 114i are arranged in concentric rings 116i (FIG. 2), which define transverse cross-sectional dimensions (i.e., diameters) that increase with distance from a centerpoint Ci of the support member 106i. As seen in FIGS. 1 and 2, the concentric rings 116i and, thus, the permanent magnets 200i, extend 360 degrees about and entirely circumscribe the centerpoint Ci of the support member 106i.

The support member 106ii extends from the backspan 104 in generally orthogonal (perpendicular) relation thereto, whereby the support members 106i, 106ii extend (are oriented) in generally parallel relation. The support member 106ii defines (second, lower) receptacles (chambers) 114ii, which are configured to receive (second, lower) permanent magnets 200ii (FIG. 1) such that the permanent magnets 200ii are secured (embedded) within the support member 106ii. The receptacles 114ii are arranged in concentric rings 116ii (FIG. 2), which define transverse cross-sectional dimensions (i.e., diameters) that increase with distance from a centerpoint Cii of the support member 106ii. As in FIGS. 1 and 2, like the concentric rings 116i, the concentric rings 116ii and, thus, the permanent magnets 200ii, extend 360 degrees about and entirely circumscribe the centerpoint Cii of the support member 106ii.

Although shown as increasing in size with distance with the respective centerpoints Ci, Cii of the support members 106i, 106ii (i.e., such that the smallest receptacles 114i, 114ii and the smallest permanent magnets 200i, 200ii are generally aligned with the centerpoints Ci, Cii), embodiments in which the receptacles 114i, 114ii may decrease in size with distance with the centerpoints Ci, Cii (i.e., such that the largest receptacles 114i, 114ii and the largest permanent magnets 200i, 200ii are generally aligned with the centerpoints Ci, Cii) are also envisioned herein, and would not be beyond the scope of the present disclosure.

In the illustrated embodiment, the backspan 104 and the support members 106 are monolithically (unitarily, integrally) formed from a single piece of material. Embodiments in which the backspan 104 and the support members 106 may be formed as separate, discrete components of the MRI system 10 are also envisioned herein, however, and would not be beyond the scope of the present disclosure. In such embodiments, it is envisioned that the backspan 104 and the support members 106 may be secured (connected) in any suitable manner (e.g., via an adhesive, via mechanical fasteners, via ultrasonic welding, etc.).

While the concentric rings 116i, 116ii are illustrated as being generally annular (e.g., circular) in configuration, it is envisioned that the particular configuration of the concentric rings 116i, 116ii may be varied. For example, embodiments in which the concentric rings 116i, 116ii may be generally elliptical in configuration are also envisioned herein, however, as are embodiments in which the concentric rings 116i, 116ii may be generally polygonal (e.g., generally square or generally rectangular) in configuration, and would not be beyond the scope of the present disclosure.

The support arms 108 are secured (connected) to the backspan 104 and/or the support members 106 and are configured to increase the strength (e.g., the rigidity) of the MRI system 10.

In the illustrated embodiment, the support arms 108 are configured as separate, discrete components of the MRI system 10 that may be secured (connected) to the backspan 104 and/or the support members 106 in any suitable manner (e.g., via an adhesive, via mechanical fasteners, via ultrasonic welding, etc.). Embodiments in which the support arms 108 may be monolithically (unitarily, integrally) formed with the backspan 104 and/or the support members 106 (i.e., such that the entire frame 100 is formed from a single piece of material) are also envisioned herein, however, and would not be beyond the scope of the present disclosure.

With reference now to FIGS. 3-5 as well, the permanent magnets 200 will be discussed. Configuring the MRI system 10 such that the support members 106 each include a plurality of discrete permanent magnets 200 (i.e., in contrast to a single permanent magnet) allows the uniformity and/or the accuracy of the magnetic field (e.g., the strength thereof) generated by the MRI system 10 to be increased (e.g., optimized) and facilitates geometric diversity. More specifically, including a plurality of permanent magnets 200 allows the configuration(s) and/or the sizes(s) (transverse cross-sectional dimension(s)) thereof to be varied, thereby altering the magnetic field.

The permanent magnets 200 are positioned (located) within the receptacles 114 defined by the support members 106, whereby the permanent magnets 200i, 200ii are respectively arranged into the aforementioned concentric rings 116i, 116ii (FIG. 2), and include configurations corresponding to (matching, mirroring) those defined by the receptacles 114. More specifically, the permanent magnets 200i are positioned (located) within the receptacles 114i defined by the support member 106i, and the permanent magnets 200ii are positioned (located) within the receptacles 114ii defined by the support member 106ii.

It is envisioned that the permanent magnets 200 may be secured within the receptacles 114 in any manner suitable for the intended purpose of inhibiting (if not entirely preventing) relative movement between the permanent magnets 200 and the support members 106. For example, in the illustrated embodiment, the permanent magnets 200 are adhesively secured within the receptacles 114. Embodiments in which the permanent magnets 200 may be mechanically secured within the receptacles 114 (e.g., via one or more mechanical fasteners, via custom plates, etc.), either in addition to or instead of an adhesive connection, are also envisioned herein, however, and would not be beyond the scope of the present disclosure.

In the illustrated embodiment, each of the permanent magnets 200 includes a generally annular (e.g., circular) transverse cross-sectional configuration, which increases the ratio of magnetic material (i.e., provided by the permanent magnets 200) to non-magnetic material (i.e. provided by the support members 106) and, thus, the strength of the magnetic field generated during operation of the MRI system 10. It is envisioned that the particular configuration of the permanent magnets 200 may be varied in alternate embodiments of the present disclosure (e.g., depending upon the necessary or desired magnitude and/or shape of the magnetic field generated during operation of the MRI system 10). For example, embodiments in which the permanent magnets 200 may include spherical configurations, cuboid configurations, elliptical configurations, etc., either exclusively or in combination, are also envisioned herein and would not be beyond the scope of the present disclosure.

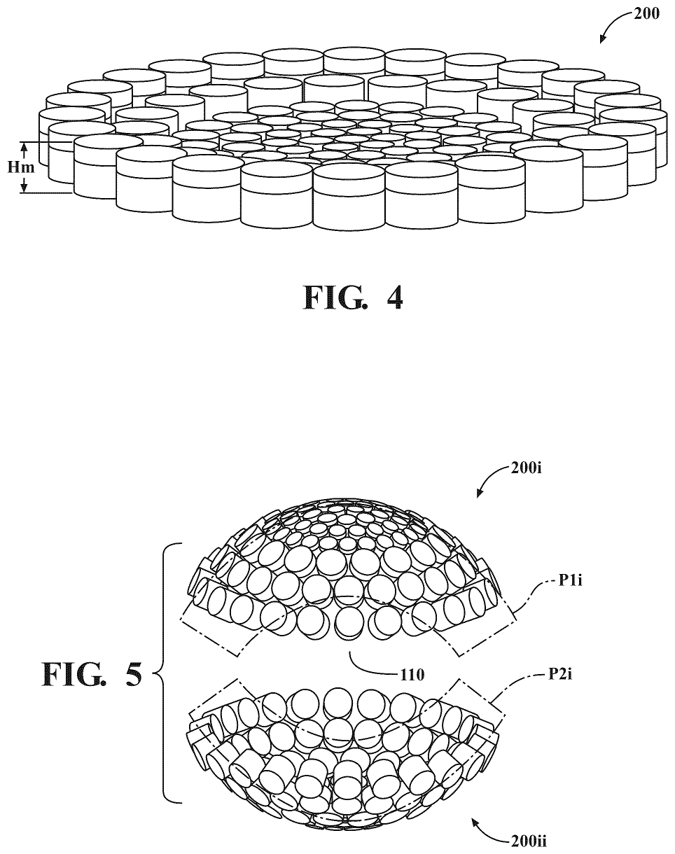

In the embodiment of the MRI system 10 illustrated in FIG. 1, the permanent magnets 200 are generally uniform in height and each defines a generally identical height Hm. FIG. 4 illustrates an alternate embodiment, however, in which the permanent magnets 200 include non-identical heights Hm that increase with (radial) distance from the centerpoints C (FIG. 2) of the support members 106 (i.e., such that the permanent magnets 200 vary in height with distance from the centerpoints C).

Additionally, while the support members 106 are configured such that the permanent magnets 200 are arranged in a generally planar orientation in the embodiment of the MRI system 10 illustrated in FIG. 1, as indicated above, in alternate embodiments, it is envisioned that the support members 106 may be configured such that the permanent magnets 200 are arranged in a non-planar orientation (i.e., such that the planes P1, P2 are curved hyperplanes) (e.g., depending upon the necessary or desired magnitude and/or shape of the magnetic field generated by the MRI system 10 during operation). For example, FIG. 5 illustrates an embodiment in which the permanent magnets 200i, 200ii are arranged in a generally spherical orientation along respective (single) hemispherical hyperplanes planes P1i, P2i, and FIG. 6 illustrates an embodiment in which the permanent magnets 200i, 200ii are arranged in a generally cylindrical orientation along respective (single) hemicylindrical hyperplanes planes P1ii, P2ii.

The permanent magnets 200i, 200ii are generally identical to each other and are non-uniform in configuration in that the permanent magnets 200i, 200ii increase in size (transverse cross-sectional dimension) with (radial) distance from the respective centerpoints Ci, Cii of the support members 106i, 106ii, as seen in FIG. 1. More specifically, in the illustrated embodiment, the permanent magnets 200i, 200ii increase in size (transverse cross-sectional dimension) uniformly with (radial) distance from the respective centerpoints Ci, Cii (FIG. 2) of the support members 106i, 106ii such that the respective permanent magnets 200i, 200ii included in the adjacent concentric rings 116i, 116ii vary in size (transverse cross-sectional dimension) by a predetermined, fixed percentage. Embodiments in which the permanent magnets 200i, 200ii may decrease in size with distance with the centerpoints Ci, Cii are also envisioned herein, however, as are embodiments in which the permanent magnets 200i, 200ii may non-uniformly vary in size with distance from the respectively centerpoints Ci, Cii of the support members 106i, 106ii. For example, embodiments are envisioned in which the receptacles 114i, 114ii and the permanent magnets 200i, 200ii may increase in size and then decrease in size with distance from the respective centerpoints Ci, Cii of the support members 106i, 106ii as are embodiments in which the permanent magnets 200i, 200ii decrease in size and then increase in size with distance from the centerpoints Ci, Cii of the support members 106i, 106ii, and would not be beyond the scope of the present disclosure. Embodiments in which the permanent magnets 200i, 200ii may be uniform in configuration (i.e., such that each of the permanent magnets 200i, 200ii is generally equivalent in size (transverse cross-sectional dimension)) are also envisioned herein, as are embodiments in which the configurations of the permanent magnets 200i may be non-identical to those of the permanent magnets 200ii. For example, embodiments in which the permanent magnets 200i and/or the permanents magnets 200ii include a variety of configurations would not be beyond the scope of the present disclosure.

In certain embodiments, such as that illustrated in FIG. 1, it is envisioned that the MRI system 10 may further include (or be otherwise connected to) a controller 118 that is configured to operate and control the MRI system 10 (e.g., the magnetic field generated by the permanent magnets 200). The controller 118 may include any component or combination of components that are suitable for the intended purpose of operating and controlling the MRI system 10 (e.g., a mobile phone, a computing device, etc.). In addition to operating and controlling the MRI system 10, it is envisioned that the controller 118 may be configured to receive and/or process diagnostic images generated during operation of the MRI system 10.

In the illustrated embodiment, the controller 118 includes: a central processing unit (CPU) 120; a memory unit 122; an output device 124 (e.g., a display 126); an imaging device 128 (e.g., a camera 130); and a microphone 132 (or other such audio component).

The CPU 120 may include any device(s) and/or components that are suitable for the intended purposes of manipulating and/or processing information. Although shown as including a single CPU 120, embodiments of the MRI system 10 that include multiple CPUs 120 (e.g., to increase the speed and/or efficiency of calculation) are also envisioned herein and would not be beyond the scope of the present disclosure.

The memory unit 122 may include a read-only memory (ROM) device, a random-access memory (RAM) device, or any other such suitable storage medium. It is envisioned that the memory unit 122 may include code and data that is accessed by the CPU 120 (e.g., using a bus). Additionally, or alternatively, it is envisioned that the memory unit 122 may include an operating system and one or more applications (programs) (e.g., an image processing application that is utilized to view, enhance, and/or process images that are generated during operation of the MRI system 10).

In order to optimize performance of the MRI system 10, it is envisioned that machine learning (e.g., AI, deep learning, etc.) may be employed (e.g., in an electromagnetic field simulation), as described in further detail below. For example, by varying the number of permanent magnets 200, the spacing between the permanent magnets 200, the specific material(s) used in construction of the permanent magnets 200 and/or the frame 100, the dimensions of the diagnostic space 110, the specific configuration(s) of the permanent magnets 200, the orientation (distribution) of the permanent magnets 200, etc., it is envisioned that the uniformity, the accuracy, the magnitude, and/or the shape of the magnetic field generated during operation of the MRI system 10 may be altered and/or that magnetic flux leakage may be reduced (if not entirely eliminated).

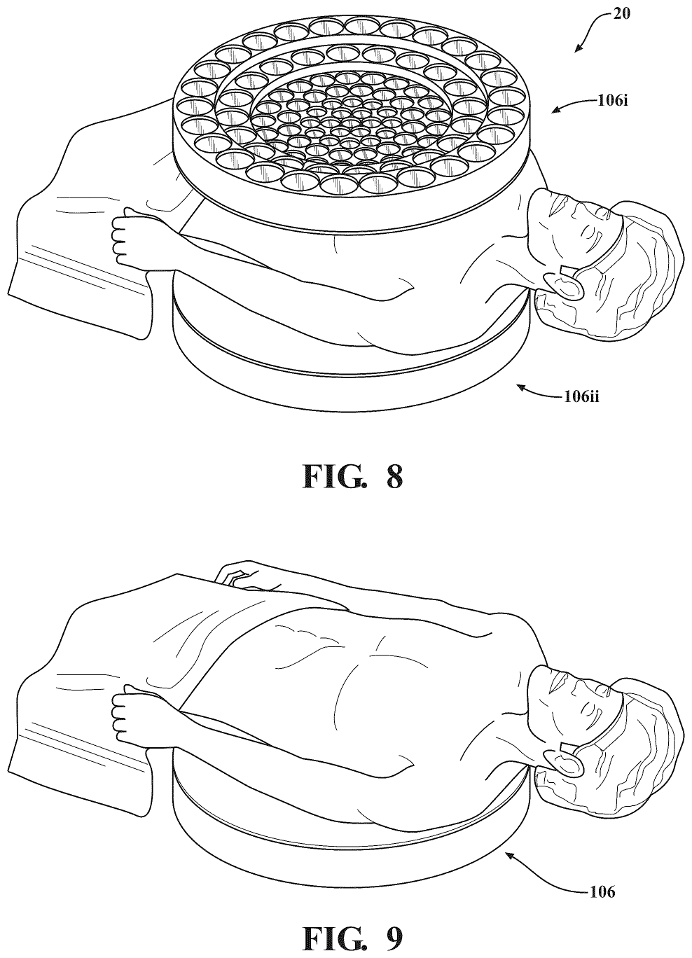

With reference now to FIGS. 7 and 8, another embodiment of the MRI system 10 is illustrated, which is identified by the reference character 20. The MRI system 20 is substantially similar to the MRI system 10 (FIG. 1) and, accordingly, will only be discussed with respect to any differences therefrom in the interest of brevity. As such, identical reference characters will be utilized to refer to elements, structures, features, etc., common to MRI systems 10, 20.

In addition to the support members 106i, 106ii, the MRI system 20 includes (first, upper and second, lower) gradient panels 134i, 134ii, which are respectively secured (connected) to, or otherwise supported by, inner surfaces 136i, 136ii of the support members 106i, 106ii, whereby the gradient panels 134i, 134ii are separated (spaced) from the permanent magnets 200i, 200ii (i.e., along an axis X that extends in generally parallel relation to the distance S). The gradient panels 134i, 134ii are configured to create spatially dependent magnetic fields through varying gradients, which supports magnetic field generation and facilitates the creation of anatomical reconstructions with accurate spatial relationships, and may include any components suitable for that intended purpose including, for example, (one or more) at least one coil 138 (e.g., RF coils, shimming coils, etc.).

Although shown as being generally planar (flat) in configuration, embodiments in which the gradient panels 134 may be arcuate (curved) are also envisioned herein (i.e., when utilized in conjunction with support members 106 that are configured to define generally spherical (FIG. 5) or generally cylindrical (FIG. 6) diagnostic spaces 110).

In the embodiment illustrated in FIGS. 7 and 8, the support members 106i, 106ii are shown as being disconnected from each other. It is envisioned, however, that the support members 106i, 106ii may be braced in any suitable manner. For example, it is envisioned that the support members 106i, 106ii may be connected via a bracket (or other such suitable structure) or that the support members 106i, 106ii may be braced independently (i.e., by separate structures).

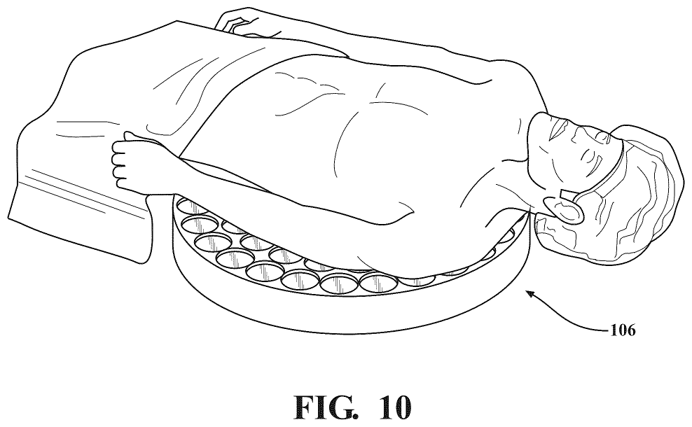

Although shown as including a pair of support members 106 in FIGS. 7 and 8 (i.e., such that a patient is positionable within the diagnostic space 110 defined therebetween), embodiments of the MRI system 20 that include a single support member 106 are also envisioned herein, as seen in FIG. 9, as are embodiments that are devoid of the gradient panels 134, as seen in FIG. 10, and would not be beyond the scope of the present disclosure.

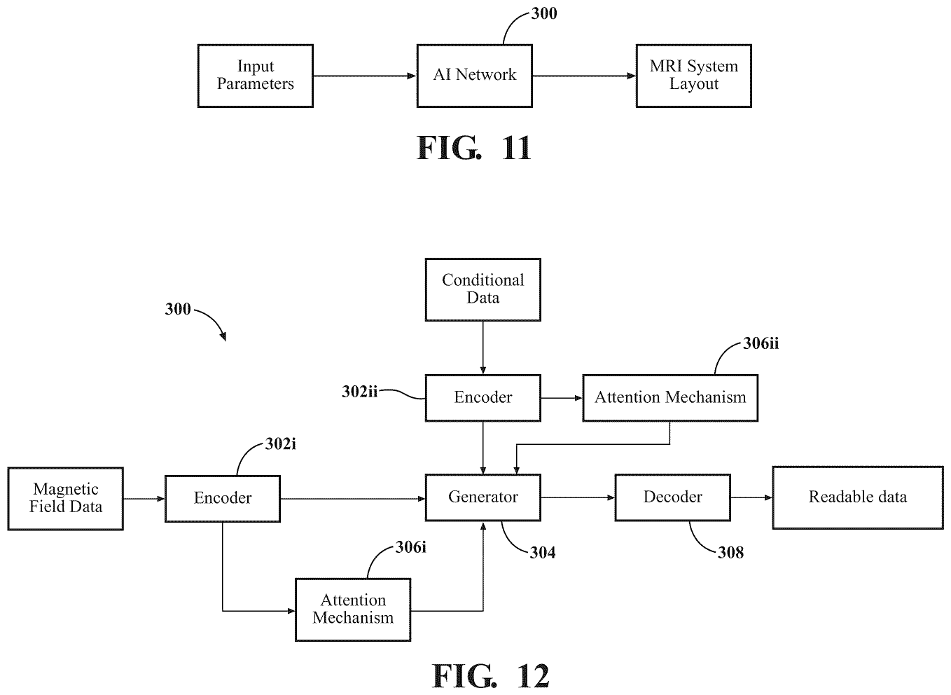

With reference now to FIG. 12, a flow diagram is provided that illustrates the use of a (generative) AI network 300 to optimize performance of the MRI system 10 (FIG. 1), 20 (FIGS. 7, 8). The AI network 300 generates a variety of different layouts (configurations) for the MRI system 10 (FIG. 1), (FIGS. 7, 8) 20 (e.g., arrangements of the permanent magnets 200) based upon various input parameters. For example, it is envisioned that one or more of the following input parameters may be utilized to generate a layout (configuration) for the MRI system 10, 20: layout boundaries for the frame 100 (FIG. 1); a maximum envelope of the MRI system 10, 20; upper and lower limits for the mass and/or the volume of the MRI system 10, 20 (e.g., the frame 100, the support members 106, the permanent magnets 200, etc.); the size of the permanents magnets 200 (e.g., expressed in terms of the heights H (FIG. 1) of the permanent magnets 200 and/or magnetic flux, such as, for example, not to exceed 125 Wb, 250 Wb, 500 Wb, etc.); the weights of the permanents magnets 200; the configurations of the permanent magnets 200 (e.g., cylindrical, cuboid, trapezoidal, etc.); the material(s) utilized in construction of the permanent magnets 200 (e.g., neodymium iron boron, samarium cobalt); the configurations of the gradient panels 134 (FIGS. 7, 8); the distance S (FIGS. 1, 7) between the support members 106; a maximum cross-sectional dimension (e.g., a diameter) of the diagnostic space 110 (e.g., not less than 200 mm); a maximum strength of magnetic field (e.g., not less than 75 mT); the uniformity of the magnetic field; uniformity in the distribution of the permanent magnets 200 (e.g., not to exceed 500 ppm); the configurations of the support members 106 (e.g., generally planar (FIGS. 1-4), generally spherical (FIG. 5), generally cylindrical (FIG. 6), etc.); regularity of the permanent magnets 200 (i.e., whether the permanent magnets 200 are uniform in size (e.g., transverse cross-sectional dimension and/or height H), increase in size with distance with the centerpoints C of the support members 106, decrease in size with distance with the centerpoints C of the support members 106, increase then decrease in size with distance with the centerpoints C of the support members 106, decrease then increase in size with distance with the centerpoints C of the support members 106, etc.). Using the AI network 300, the amount of required magnetic material can be reduced (e.g., minimized), thereby reducing the overall cost of the MRI system 10, 20.

The AI network 300 integrates electromagnetic field simulation with advanced machine learning (such as deep learning) techniques that are tailored to the particular demands of the MRI system 10 in order to facilitate design of the permanent magnets 200 and the MRI system 10 as a whole in order to improve the precision and efficiency thereof. More specifically, the AI network 300 can be developed (trained) by mapping the magnetic field of the MRI system 10 to various permanent magnets 200 using the input parameters. The AI network 300 then dynamically generates layouts and configurations for the MRI system 10 (e.g., the permanent magnets 200).

In certain embodiments, it is envisioned that the AI network 300 may include a model that is trained to output data for the permanent magnets 200 (e.g., structure, layout, etc.) based the input parameter(s) (e.g., magnetic field data). For example, following the generation of a layout for the MRI system 10, 20 (e.g., the permanent magnets 200), a magnetic field generation software tool can be utilized to perform a magnetic field simulation to evaluate the efficiency of the layout by comparing the generated magnetic field to a target magnetic field. The input parameter(s) can then be modified as necessary to alter the layout for the MRI system 10, 20 and, thus, the magnetic fields generated thereby.

With reference to FIG. 12, in one embodiment of the disclosed methodology, certain input parameter(s) (e.g., magnetic field data) is input into a (first) encoder 302i, and certain input parameter(s) (e.g., conditional data and other constraints concerning the configuration of the permanent magnets 200) is input into a (second) encoder 302ii, which respectively refine and encode the magnetic field data and the conditional data. The encoders 302i, 302ii then respectively output the encoded magnetic field data and the encoded conditional data to a generator 304.

Although shown as utilizing two separate encoders 302i, 302ii, embodiments are also envisioned in which a single encoder 302 may be utilized. In such embodiments, it is envisioned that the (single) encoder 302 may receive both the magnetic field data and the conditional data.

In various embodiments of the AI network 300, it is envisioned that attention mechanisms 306i, 306ii may be utilized to orchestrate and refine the data that is input into the generator 304, as seen in FIG. 6.

The generator 304 outputs a representative model of the permanent magnets 200 to a decoder 308, which decodes the representative model and outputs readable data concerning the configuration of the permanent magnets 200.

It is envisioned that certain embodiments of the disclosure may be implemented as methods, systems, computer programs, hardware and/or software implementations, etc.

Additionally, it is envisioned that certain embodiments of the disclosure may be embodied in one or more (executable) computer programs, which may be stored in any suitable medium (e.g., disk storage, optical storage, etc.). In such embodiments, it is envisioned that the included instructions may be provided to generic computers, special-purpose computers, embedded computers, or any other suitable processor of programmable data.

It is envisioned that such computer programs may be stored in any suitable computer-readable storage medium that is able to boot a computer (or other such programmable data processing device) to a specific work mode, and that the stored instructions may produce a manufactured product that implements various functions. Additionally, or alternatively, it is envisioned that such computer programs may be loaded onto a computer (or other such programmable data processing device) to execute a series of operating procedures and produce a process that is implemented by the computer, whereby the computer program provides operating procedures for various functions.

Persons skilled in the art will understand that the various embodiments of the disclosure described herein and shown in the accompanying figures constitute non-limiting examples, and that additional components and features may be added to any of the embodiments discussed herein above without departing from the scope of the present disclosure. Additionally, persons skilled in the art will understand that the elements and features shown or described in connection with one embodiment may be combined with those of another embodiment without departing from the scope of the present disclosure and will appreciate further features and advantages of the presently disclosed subject matter based on the description provided. Variations, combinations, and/or modifications to any of the embodiments and/or features of the embodiments described herein that are within the abilities of a person having ordinary skill in the art are also within the scope of the disclosure, as are alternative embodiments that may result from combining, integrating, and/or omitting features from any of the disclosed embodiments.

Use of broader terms such as “comprises,” “includes,” and “having” should be understood to provide support for narrower terms such as “consisting of,” “consisting essentially of,” and “comprised substantially of.” Accordingly, the scope of protection is not limited by the description set out above but is defined by the claims that follow and includes all equivalents of the subject matter of the claims.

In the preceding description, reference may be made to the spatial relationship between the various structures illustrated in the accompanying drawings, and to the spatial orientation of the structures. However, as will be recognized by those skilled in the art after a complete reading of this disclosure, the structures described herein may be positioned and oriented in any manner suitable for their intended purpose. Thus, the use of terms such as “above,” “below,” “upper,” “lower,” “inner,” “outer,” “left,” “right,” “upward,” “downward,” “inward,” “outward,” etc., should be understood to describe a relative relationship between the structures and/or a spatial orientation of the structures. Those skilled in the art will also recognize that the use of such terms may be provided in the context of the illustrations provided by the corresponding figure(s).

Additionally, terms such as “approximately,” “generally,” “substantially,” and the like should be understood to allow for variations in any numerical range or concept with which they are associated and encompass variations on the order of 25% (e.g., to allow for manufacturing tolerances and/or deviations in design). For example, the term “generally parallel” should be understood as referring to configurations in with the pertinent components are oriented so as to define an angle therebetween that is equal to 180°±25% (i.e., an angle that lies within the range of (approximately) 135° to (approximately) 225°) and the term “generally orthogonal” should be understood as referring to configurations in with the pertinent components are oriented so as to define an angle therebetween that is equal to 90°±25% (i.e., an angle that lies within the range of (approximately) 67.5° to (approximately) 112.5°). The term “generally parallel” should thus be understood as referring to encompass configurations in which the pertinent components are arranged in parallel relation, and the term “generally orthogonal” should thus be understood as referring to encompass configurations in which the pertinent components are arranged in orthogonal relation.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present disclosure.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A magnetic resonance imaging (MRI) system comprising:
a support member; and
permanent magnets positioned within the support member such that the permanent magnets are arranged along a single plane, wherein the permanent magnets vary size.

2. The MRI system of claim 1, wherein the permanent magnets are arranged such that the single plane is linear.

3. The MRI system of claim 2, wherein the permanent magnets are arranged in concentric rings.

4. The MRI system of claim 1, wherein the permanent magnets are arranged such that the single plane is a curved hyperplane.

5. The MRI system of claim 4, wherein the curved hyperplane is hemispherical.

6. The MRI system of claim 4, wherein the curved hyperplane is hemicylindrical.

7. The MRI system of claim 1, wherein the permanent magnets increase in size with distance from a centerpoint of the support member.

8. The MRI system of claim 7, wherein the permanent magnets increase in transverse cross-sectional dimension with distance from the centerpoint of the support member.

9. The MRI system of claim 8, wherein the permanent magnets are generally uniform in height.

10. The MRI system of claim 8, wherein the permanent magnets increase in height with distance from the centerpoint of the support member.

11. A magnetic resonance imaging (MRI) system comprising:
a support member including a non-magnetic, low-conductivity material; and
permanent magnets positioned within the support member in a uniform, symmetrical distribution.

12. The MRI system of claim 11, wherein the permanent magnets include generally annular transverse cross-sectional configurations.

13. The MRI system of claim 11, wherein the permanent magnets vary in size.

14. The MRI system of claim 13, wherein the permanent magnets vary in transverse cross-sectional dimension with distance from a centerpoint of the support member.

15. The MRI system of claim 14, wherein the permanent magnets vary in height with distance from the centerpoint of the support member.

16. A magnetic resonance imaging (MRI) system comprising:
a first support member;
first permanent magnets positioned within the first support member, wherein the first permanent magnets vary in size;
a second support member facing the first support member, wherein the first support member and the second support member each include a non-magnetic, low-conductivity material; and
second permanent magnets positioned within the second support member, wherein the second permanent magnets vary in size.

17. The MRI system of claim 16, wherein the first support member and the second support member are generally identical in configuration, and wherein the first permanent magnets and the second permanent magnets are generally identical in configuration.

18. The MRI system of claim 16, wherein the first permanent magnets increase in transverse cross-sectional dimension with distance from the centerpoint of the first support member, and the second permanent magnets increase in transverse cross-sectional dimension with distance from the centerpoint of the second support member.

19. The MRI system of claim 16, wherein the first permanent magnets increase in height with distance from a centerpoint of the first support member, and the second permanent magnets increase in height with distance from a centerpoint of the second support member.

20. The MRI system of claim 16, further comprising:
a first gradient panel secured to the first support member; and
a second gradient panel secured to the second support member, wherein the first gradient panel and the second gradient panel each include at least one coil configured to support magnetic field generation.
